(19) United States
(12) Patent Application Publication
(10) Pub. No.: US 2013/0046166 A1
(43) Pub. Date: Feb. 21, 2013

(54) INTRAOCULAR PRESSURE MONITORING SYSTEM

(71) Applicants: Teimour Maleki Jafarabadi, West Lafayette, IN (US); Babek Ziaie, West Lafayette, IN (US); Girish Chitnis, West Lafayette, IN (US); Louis B. Cantor, Indianapolis, IN (US)

(72) Inventors: Teimour Maleki Jafarabadi, West Lafayette, IN (US); Babek Ziaie, West Lafayette, IN (US); Girish Chitnis, West Lafayette, IN (US); Louis B. Cantor, Indianapolis, IN (US)

(73) Assignee: PURDUE RESEARCH FOUNDATION, West Lafayette, IN (US)

(21) Appl. No.: 13/639,886

(22) PCT Filed: Apr. 6, 2011
(86) PCT No.: PCT/US2011/031437

§ 371 Date: Oct. 8, 2012

Related U.S. Application Data
(60) Provisional application No. 61/321,494, filed on Apr. 6, 2010.

(51) Int. Cl. A61B 3/16

(52) U.S. Cl. 600/398

(57) ABSTRACT

A pressure monitoring system is disclosed. The pressure monitoring system includes a stimulation/response circuit configured to transmit a first signal during a stimulation mode and receive a second signal during a response mode, and a pressure monitoring apparatus configured to receive the first signal and transmit the second signal. The pressure monitoring apparatus includes a capacitive pressure sensor assembly, an insertion needle assembly, and a coil assembly. The insertion needle assembly, having an insertion opening, is coupled to the capacitive pressure sensor assembly, wherein fluid pressure at the insertion opening is fluidly communicated with the capacitive pressure sensor assembly. The coil assembly, having an inductance, is coupled to the capacitive pressure sensor assembly, wherein the coil assembly and the capacitive pressure sensor assembly form a tank circuit with a variable resonant frequency, and wherein the coil assembly receives the first signal and transmits the second signal a time difference later.

### PRIORITY

This application claims the benefit of a U.S. Provisional Application Ser. No. 61/321,494 the entire content of which is incorporated herein by reference.

### TECHNICAL FIELD

The present invention generally relates to pressure monitoring devices and particularly to pressure monitoring devices for monitoring pressure within a biological system.

### BACKGROUND

Intraocular pressure (TOP) monitoring is essential in study and cure of diseases such as glaucoma. Increased and decreased IOP both are potentially harmful to a patient's eyesight. In many cases the damage caused is irreversible. Hence it is important to monitor the IOP continuously and accurately in patient with a diseased eye. So far there have been devices which attempt to measure the IOP based on applied pressure and deformation of the eyeball. However these devices are bulky, and are not capable of continuous monitoring.

Therefore, there is a need for a system for monitoring of the IOP in a patient's eye with a minimally invasive device which is light weight, portable, and capable of providing continuous measurements and communicating the measurement to an external monitoring device.

### SUMMARY

According to one aspect of the current teachings a pressure monitoring system is disclosed. The pressure monitoring system includes a stimulation/response circuit configured to transmit a first signal during a stimulation mode, and receive a second signal during a response mode in response to the first signal, and a pressure monitoring apparatus configured to receive the first signal during the stimulation mode and transmit the second signal during the response mode, wherein the pressure monitoring apparatus includes a capacitive pressure sensor assembly providing a variable capacitance in response to variable pressure being applied to the capacitive pressure sensor assembly, an insertion needle assembly having an insertion opening, the insertion needle assembly being coupled to the capacitive pressure sensor assembly, wherein fluid pressure at the insertion opening is fluidly communicated with the capacitive pressure sensor assembly thereby affecting capacitance of the capacitive pressure sensor assembly, and a coil assembly having an inductance, the coil assembly being coupled to the capacitive pressure sensor assembly, wherein the coil assembly and the capacitive pressure sensor assembly form a tank circuit with a variable resonant frequency, and wherein the coil assembly receives the first signal and transmits the second signal a time difference later.

According to another aspect of the current teachings a pressure monitoring apparatus is disclosed. The pressure monitoring apparatus includes a capacitive pressure sensor assembly providing a variable capacitance in response to variable pressure being applied to the capacitive pressure sensor assembly, an insertion needle assembly having an insertion opening, the insertion needle assembly being coupled to the capacitive pressure sensor assembly, wherein fluid pressure at the insertion opening is fluidly communicated with the capacitive pressure sensor assembly thereby affecting capacitance of the capacitive pressure sensor assembly, and a coil assembly having an inductance, the coil assembly being coupled to the capacitive pressure sensor assembly.

According to yet another aspect of the current teachings a method for monitoring intraocular pressure is disclosed. The method includes implanting a pressure monitoring apparatus in a subject's eye, the pressure monitoring apparatus having a variable capacitance in response to intraocular pressure, and an inductance forming a tank circuit, fluidly coupling intraocular fluid with the pressure monitoring apparatus, transmitting a first signal during a stimulation mode, receiving the first signal by the tank circuit during the stimulation mode, energizing the tank circuit in response to receiving the first signal, and transmitting a second signal by the tank circuit a period of time after receiving the first signal.

### BRIEF DESCRIPTION OF DRAWINGS

FIG. 1 is a schematic view of an intraocular pressure monitoring system, including a stimulation/response circuit and an intraocular pressure monitoring apparatus (IPMA), according to the present disclosure.

FIG. 2 is a perspective view of the IPMA of FIG. 1 including various components of the IPMA.

FIG. 3 is bottom view of a coil assembly of the IPMA of FIG. 2.

FIG. 4 is a perspective view of a capacitor assembly of the IPMA of FIG. 2.

FIG. 5 is a perspective view of a needle assembly of the IPMA of FIG. 2.

FIG. 6 is a schematic of the stimulation/response circuit of FIG. 1 positioned near the IPMA of FIG. 2.

FIG. 7 is a schematic view of various implantation sites in an animal.

FIG. 8 is a graph of pressure vs. resonant frequency of the IPMA of FIG. 2 achieved in laboratory environment.

FIG. 9 is a graph of frequency vs. phase obtained for an IPMA in animal studies.

FIG. 10 is a perspective view of a delivery apparatus for implanting the IPMA of FIG. 2 at various sites of an eye.

FIGS. 11-I, 11-II, 11-III, and 11-IV are cross sectional views showing fabrication steps of the IPMA of FIG. 2.

### DETAILED DESCRIPTION

For the purposes of promoting an understanding of the principles of the invention, reference will now be made to the embodiments illustrated in the drawings and described in the following written specification. It is understood that no limitation to the scope of the invention is thereby intended. It is further understood that the present invention includes any alterations and modifications to the illustrated embodiments and includes further applications of the principles of the invention as would normally occur to one of ordinary skill in the art to which this invention pertains.

A system for using a capacitive pressure sensor for monitoring intraocular pressure (TOP) changes is disclosed. This type of sensor provides advantages of high sensitivity and low power consumption. In addition, zero DC power consumption and convenient inductor-capacitor (L-C) tank wireless readout circuitry make capacitive pressure sensors more favorable for this type of application.

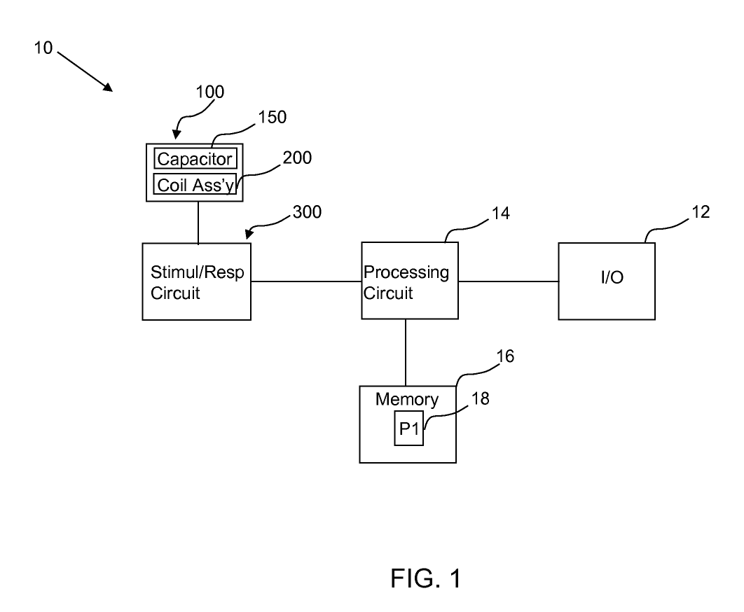

FIG. 1 depicts an TOP measuring system 10. The IOP measuring system 10 includes an

I/O device 12, a processing circuit 14 and a memory 16. The I/O device 12 may include an input/output user interface, graphical user interface, keyboards, pointing devices, remote and/or local communication links, displays, and other devices that allow externally generated information to be provided to the TOP measuring system 10, and also allow internal information of the IOP measuring system 10 to be communicated externally.

The processing circuit 14 may suitably be a general purpose computer processing circuit such as a microprocessor and its associated circuitry. Alternatively, the processing circuit may be a dedicated instrumentation equipment specifically designed to operate the TOP measuring system 10. The processing circuit 14 is operable to carry out operations related to measuring the IOP pressure. The processing circuit 14 is connected to the I/O circuit 12 for receiving information from the I/O circuit 12 and for providing information to the I/O circuit 12.

The memory 16 stores program instructions 18 that are executed by the processing circuit 14 and/or any other components as appropriate. The memory 16 is also configured to store data from the IOP measurements as well as other data related to the IOP measuring system 10. The memory 16 may include read-only memory (ROM), as well as electrically erasable programmable ROM, random access memory and other forms of memory known by a person of ordinary skill in the art. The memory 16 is connected to the processing circuit 14 to provide information to the processing circuit 14 as well as receive information from the processing circuit 14.

The IOP measuring system 10 further includes a stimulation/response circuit 300 connected to the processing circuit 14. The sensor stimulation/response circuit 300 provides a stimulus for an intraocular pressure monitoring apparatus (IPMA) 100 and measures the effects of the stimulus. The stimulus may be controlled by the processing circuit 14 and where the measured value is communicated to the processing circuit 14. The IPMA 100 includes the capacitive pressure sensor assembly 150 as well as a coil assembly 200, described in greater detail below.

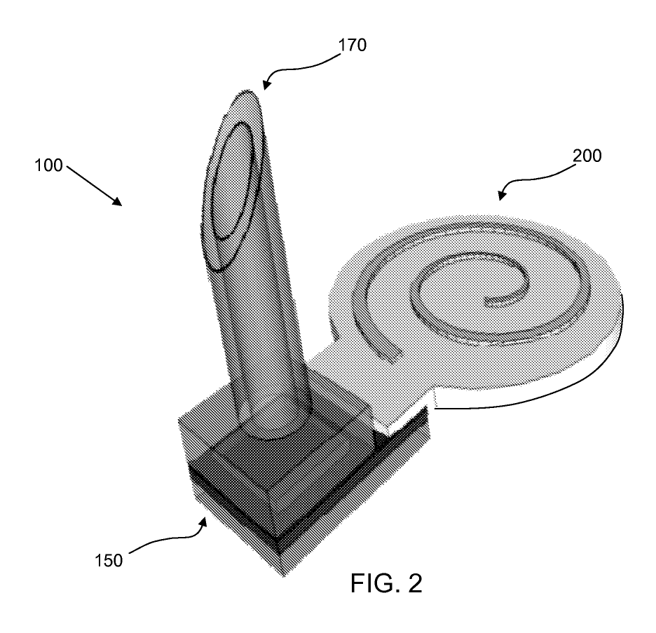

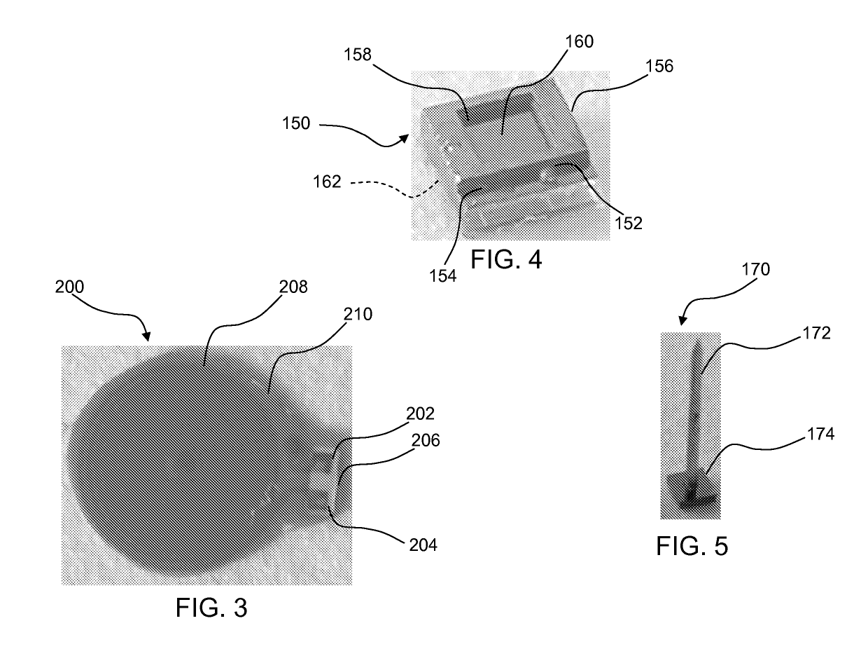

Referring to FIG. 2, a perspective view of an exemplary design of an IPMA 100 is provided. The IPMA 100 includes a capacitive pressure sensor assembly 150, an insertion needle assembly 170, and a coil assembly 200. The insertion needle assembly 170 is mechanically coupled to the capacitive pressure sensor assembly 150. The mechanical coupling between the capacitive pressure sensor assembly 150 and the insertion needle assembly 170 provides a fluid path from a portion of an eye through the insertion assembly on to a surface of the capacitive pressure sensor assembly 150, as described further below. The mechanical coupling can be achieved by an adhesive or as explained below by anodic bonding. Alternatively, the insertion needle assembly 170 and the capacitive pressure sensor assembly 150 can be formed integrally, as described below. Also, the coil assembly 200 and the capacitive pressure sensor assembly 150 are mechanically coupled to each other. The mechanical coupling between the capacitive pressure sensor assembly 150 and the coil assembly 200 provides an electrical path from the capacitive pressure sensor assembly 150 to the coil assembly 200, as described further below. The mechanical coupling can be achieved by an adhesive. In addition to the mechanical coupling, the coil assembly 200 includes two electrical terminals (depicted in FIG. 3, discussed below) that are brought to contact with respective electrical terminals on the capacitive pressure sensor assembly 150 (depicted in FIG. 4, also discussed below).

Referring to FIG. 3, a bottom view of the coil assembly 200 is depicted. The coil assembly 200 includes terminals 202 and 204 that are encased in a pad 206 made of an insulating material such as glass. The terminals 202 and 204 can be made from Ti/Au, Cr/Au, Ti/Pt, Cr/Pt,

Cu, Al and any conductive materials. Each of the two terminals 202 and 204 is connected to a wire 208 that is wrapped in the shape of a coil. The coil-shaped wire 208 is made form a conductive material such as Ti/Au, Cr/Au, Ti/Pt, Cr/Pt, Cu, Al and other conductive materials. The coil-shaped wire 208 is part of an L-C tank circuit, described further below, wherein the coil-shaped wire 208 provide the majority of the inductance. The wire 208 is encased with a bio-compatible and inert material layer 210 such as glass, parylene, polyimide, Polydimethylsiloxane (PDMS), acrylic, Cyclobutene (BCB), or other polymers. The layer 210 seals the wire 208 such that when the IPMA 100 is implanted into the eye, ocular fluids do not cause shorting of the wires between each rotation of the wire in the coil-shaped wire 208. Such a shorting can adversely affect the inductance.

Referring to FIG. 4, a perspective view of the capacitive pressure sensor assembly 150 is depicted. The capacitive pressure sensor assembly 150 includes terminals 152 and 154 disposed adjacent a housing 156. The terminals can be made from Ti/Au, Cr/Au, Ti/Pt, Cr/Pt, Cu, Al and other conductive materials. The housing 156 encapsulates a capacitor formed in a well 158. The housing can be made from silicon, glass, SU-8, acrylic, aluminum, copper, titanium and other structural material with appropriate electrical insulating qualities. The capacitor includes a top flexible membrane 160 and a lower plate 162. The top flexible membrane 160 can be made from various materials such as doped silicon. The top flexible membrane 160 is configured to be in fluid communication with ocular fluid and further configured to deflect in the presence of intraocular fluid pressure. The top flexible membrane 160 is part of a capacitor which also includes the lower plate 162. The lower plate can be made form various material such as doped silicon. The capacitor also includes a dielectric layer (not shown) disposed between the top flexible membrane 160 and the lower plate 162. The dielectric layer (not shown) can be made form silicon nitride, silicon dioxide, parylene, alumina, titania, BCB, SU-8, and other material with the appropriate electrical qualities.

The capacitor is part of the L-C tank circuit, discussed above. The capacitance of the capacitor is affected by the position of the top flexible membrane 160. In response to the flexure of the top flexible membrane 160, lump capacitance of the capacitor increases based on the following formula:

$$C = {ɛ}_{r} {ɛ}_{0} \frac{S}{d} ,$$

wherein C is the capacitance,
εᵣ is the dielectric constant (also known as the relative static permittivity),
ε₀ is the electric constant,
S is the surface area of the overlapped portions of the top flexible membrane 160 and the bottom plate 162, and
d is the distance between the top flexible membrane 160 and the bottom plate 162. The quantity
εᵣ is dependent on the material chosen for the dielectric. The capacitance C has an inverse relationship with the distance between the top flexible membrane 160 and the bottom plate 162. Therefore, as the distance d decreases the capacitance C increases. Increase in the capacitance C affects the resonant frequency of the tank circuit according to the following formula:

$$f = \frac{1}{2 π \sqrt{LC}} ,$$

wherein f is the resonant frequency of the tank circuit,
L is the inductance, and
C is the capacitance. In the IPMA 100, the inductance L is configured to be substantially constant as defined by the coil assembly 200, while the capacitance C is configured to vary in response to application of IOP. As the IOP increases, the distance between the top flexible membrane 160 and the bottom plate 162 decreases, which causes the capacitance C to increase, which in turn causes the resonant frequency f to decrease.

Referring to FIG. 5, a perspective view of the insertion needle assembly 170 is depicted. The insertion needle assembly includes a hollow needle 172 attached to a base portion 174. The hollow portion of the needle 172 is best shown in FIG. 2. The needle 172 can be made form stainless steel, glass and other biologically inert material that have a high tensile strength. The hollow portion of the needle 172 continues through the base portion 174. As a result, the insertion needle assembly 172 is configured to transfer IOP to the capacitor by fluidly coupling the ocular fluid to the top flexible membrane 160 (see FIG. 4). The base portion 174 is configured to tightly fit inside the well 158 when the insertion needle 170 is assembled with the capacitive pressure sensor assembly 150. The base portion 174 can be made from Silicon, glass, SU-8, acrylic, aluminum, copper, titanium, or other material with appropriate structural qualities. Alternatively, the insertion needle assembly 170 including the base portion 174 can be integrally formed with the capacitive pressure sensor assembly 150, as discussed below.

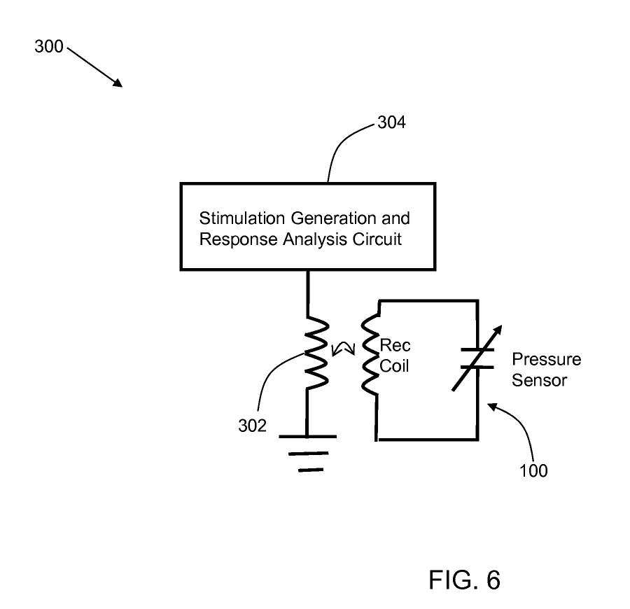

Referring to FIG. 6, a schematic view of the stimulation/response circuit 300 positioned next to the IPMA 100 is depicted. The circuit 300 includes a coil 302 that is operated in a stimulation mode as a transmission coil and also used in a response mode as a pickup coil. In the stimulation mode, the coil 302 transmits a first signal. In the response mode, the IPMA 100 retransmits a second signal in response to the transmission of the first signal. The coil 302 picks up the second signal in the response mode. It is to be appreciated that different types of stimulation/response circuits 300 can be implemented in the IOP measuring system 10. The stimulation/response circuit 300 is only required to 1) provide a transmission signal during the stimulation mode, which as described below is a frequency sweeping signal about the expected resonant frequency of the tank circuit, and 2) receive the retransmitted signal during the response mode for further analysis, such as comparing phases of the transmitted and the retransmitted signals. Accordingly, the coil 302 receives the first signal from and provides the second signal to a stimulation and response analysis circuit 304. In addition, as discussed above, the stimulation/response circuit 300 is connected to the processing circuit 14 for communication therewith. It should be appreciated that this communication can be via a wireless channel.

In operation, the transmitted signal (i.e., the first signal) that is picked up by the coil assembly 200 energizes the LC tank circuit. Depending on the frequency of the transmitted signal and how close that frequency is to the resonant frequency of the LC tank circuit (discussed above), a short amount of time later, the tank circuit begins to retransmit the second signal. The time lag between the two signals (i.e., the original transmitted signal by the coil 302 and the retransmitted signal by the tank circuit) define the phase difference between these signals. At resonant frequency magnitude of the retransmitted signal approaches the magnitude of the transmitted signal. However, due to losses in the tank circuit, mainly electrical resistance of the coil assembly 200 and contact resistance between the terminals 202 and 204 of the coil assembly 200 and the terminals 152 and 154 of the capacitive pressure sensor assembly 150, the retransmitted signal has an attenuated magnitude as compared to the transmitted signal even at the resonant frequency.

The authors of the present disclosure envision the stimulation/response circuit 300 to be mountable on a pair of glasses that can be worn by a human subject which can wirelessly (or with a wired channel) communicate with the processing circuit 14. The stimulation/response circuit 300 can be mounted on the glasses near the eye for monitoring IOP.

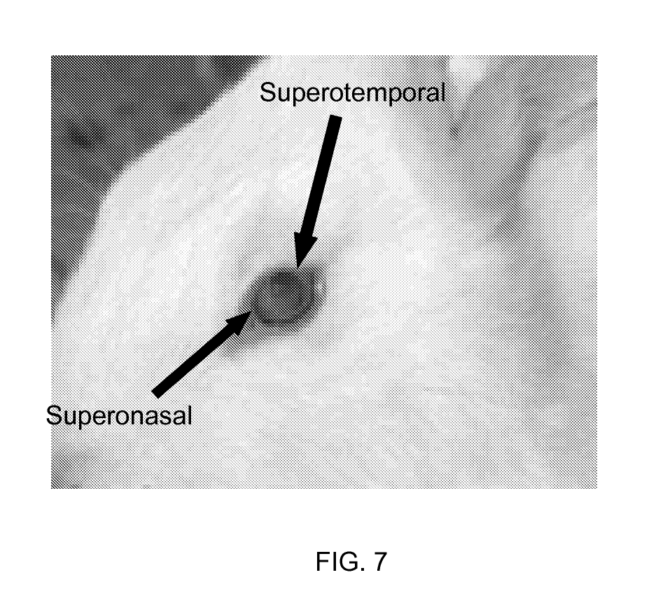

According to one embodiment, upon implantation, the needle 172 penetrates the sclera and then can be in contact with the vitreous chamber which contains the vitreous humor. Referring to FIG. 7, various sites (e.g., superotemporal or superonasal) for implantation of IPMA 100 are depicted. In either of two exemplified sites, the bulk of the IPMA 100, including the capacitive pressure sensor assembly 150 as well as the coil assembly 200 can be placed underneath the orbital fat. In animal studies, the authors of the present disclosure have shown that the IPMA 100 can be implanted in any of these sites with minimal irritation to the animal throughout the course of the study.

The authors of the present disclosure have also shown by experiments on cadaver eyes that pressure values based on measurements at the posterior part of the eye correlate well with pressure values at the anterior part of the eye making IPMA 100 relatively location independent.

Most other intra-ocular sensor technologies are designed to be totally implanted within the eye and/or to perform measurement at the anterior chamber of the eye. However with the IPMA 100, partial insertion of the sensor makes the sensor minimally invasive, easy to implant, and generates minimal irritation for the subject.

Once implanted, the insertion needle assembly 170 established fluid communication between the capacitive pressure sensor assembly 150 and the ocular fluid. Accordingly, IOP in the anterior chamber of the eye applies pressure to the top flexible membrane 160 of the capacitive pressure sensor assembly 150 causing the top flexible membrane 160 to flex inwardly toward the bottom plate 162. As discussed above. the flexure of the deformable membrane increases the capacitance of the capacitive pressure sensor assembly 150.

The stimulation/response circuit 300, provides the stimulation signal with a sweeping frequency near the expected resonant frequency of the tank circuit of the capacitive pressure sensor assembly 150 during the stimulation mode. The tank circuit is thereby energized by electromagnetically coupling the external field generated by the coil 302 (see FIG. 6). Energizing the tank circuit, causes the tank circuit to transmit its own signal during the response mode with a phase shifted retransmitted signal (i.e., a signal with a time lag) as compared to the original transmitted signal by the coil 302. The response signal is in turn picked up by the coil 302. When the frequency of the field approaches the resonant frequency of the tank circuit, the transfer function of the tank circuit approaches one and, therefore, strength of the electromagnetic field generated by the tank circuit approaches strength of the electromagnetic field that is induced by the coil 302. This increase in the field strength can be used to identify the capacitance of the capacitive pressure sensor assembly 150, which can in turn be correlated to the pressure applied to the top deformable membrane 160 (see FIG. 4).

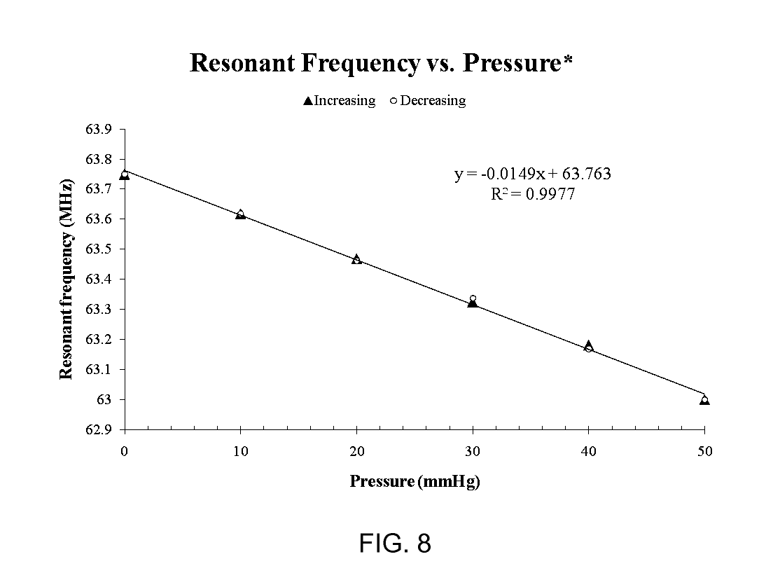

Referring to FIG. 8 a graph of TOP (in mmHg) vs. resonant frequency (in MHz) of the tank circuit obtained in laboratory settings is provided. The graph shows a linear relationship governed by:

f=−0.0149P+63.763,

wherein f is frequency of the resonant frequency of the tank circuit, and
P is the pressure applied to the top flexible membrane 160. This relationship agrees with the analysis discussed above. As the pressure increases, the top flexible membrane 160 deforms, causing the capacitance of the tank circuit to increase, which causes the resonant frequency of the tank circuit to decrease.

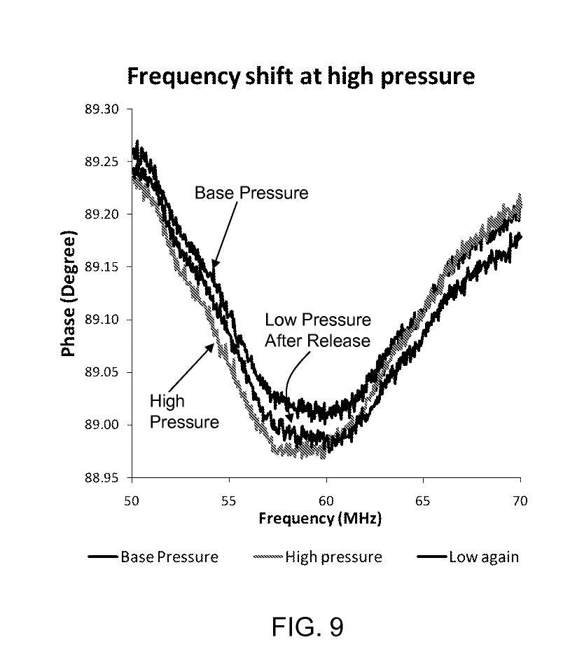

Referring to FIG. 9, a graph of frequency (in MHz) vs. phase shift (in degrees) is depicted. As discussed above, the phase difference is between the transmitted signal in the stimulation mode and the retransmitted (or received) signal in the response mode. The graph in FIG. 9 shows the relationship between frequency and phase shift for three different pressures: 1) high pressure, generated by depressing the eye; 2) base pressure, and 3) low pressure resulting after releasing the eye. In all three cases, a quadratic relationship is depicted between frequency and phase shift as frequency is swept form left to right.

In reference to FIG. 9, a narrow and deep phase change dip is desirable to increase sensitivity of the IPMA 100. If the phase change dip is wide and shallow, the sensitivity of the IPMA 100 decreases as it is affected by the stimulation/response circuit 300 (see FIG. 6, and the discussion provided above).

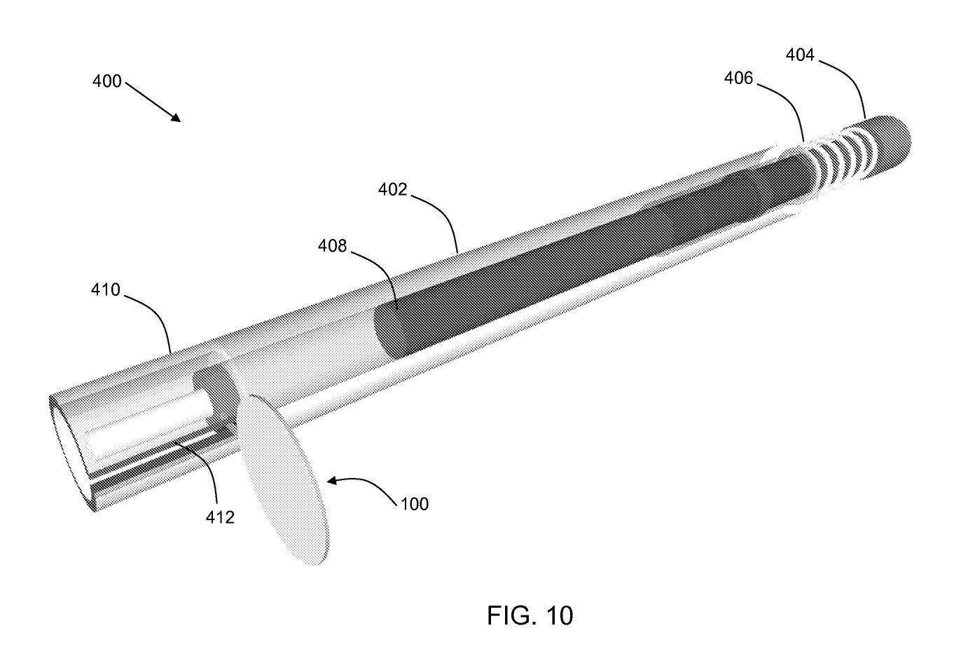

Referring to FIG. 10, an insertion tool 400 for the IPMA 100 is depicted. The insertion tool 400 includes a housing 402, an actuation knob 404, and a plunger 408 coupled to the actuation knob 404. A biasing member 406 is positioned between the actuation knob 404 and the housing 402 in order to bias the actuation knob 404 away from the housing 402. The housing 402 terminates at an actuation end 410 which is provided with a slot 412 for receiving the IPMA 100. The slot 412 is sufficiently wide in order to receive the terminal connection (as discussed above). The actuation tool 400 is shown in a non-actuated position.

In operation, a clinician/researcher prepares the desired implantation site in a subject's eye. The clinician/researcher loads an IPMA 100 into the actuation end 410 and positions the insertion tool 400 over the desired site. Once positioned, the clinician/researcher presses the actuation knob so that the plunger 408 comes in contact with IPMA 100 and causes forward motion of the IPMA 100 into the subject's eye. Upon release of the actuation knob 404, the knob 404 under the biasing force of the biasing member 406 returns to the non-actuated position (depicted in FIG. 10).

### Packaging and Fabrication

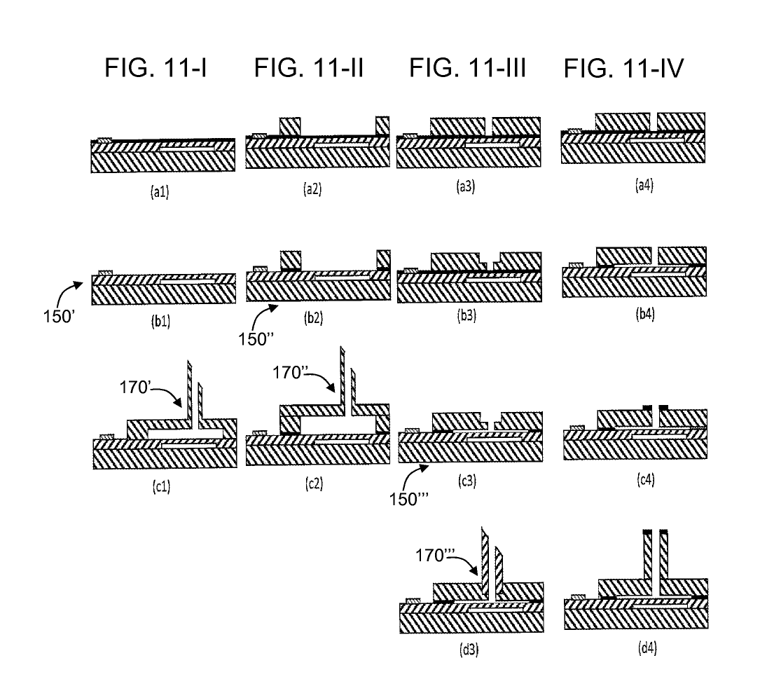

Different packaging and fabrication methods have been developed to fabricate and assemble an IPMA 100 which are described below. Process flows for these packaging methods, starting with a pressure sensor with backside silicon, is shown in FIGS. 11-I through 11-IV. In all these figures, processes are described using a silicon-on-insulator (SOI) wafer, containing handle, buried oxide and device layers that are bonded to a glass wafer

Referring to FIG. 11-I, a capacitive pressure sensor assembly 150′ and an insertion needle assembly 170′ are fabricated separately and then bonded together by a method such as anodic bonding. In this process, the handle and the buried oxide layers of the SOI wafer are removed, FIG. 11-I (a1 and b1), to generate the capacitive pressure sensor assembly 150′. Then a separately fabricated insertion needle assembly 170′ is bonded to the capacitive pressure sensor assembly 150′, FIG. 11-I (c1). Many bio-compatible materials are suitable for the insertion needle assembly 170′. However using glass or titanium would allow the fabricated insertion needle assembly 170′ to be bonded anodically to the capacitive pressure sensor assembly 150′. In case of other materials, such as steel or a polymer, epoxy glue (such as BCB) is used for bonding of housing and the pressure sensor. The housing can be fabricated using ultrasonic machining. The housing can be fabricated as an array on a glass wafer, hence the process is suitable for batch fabrication. To achieve the assembly depicted in FIG. 11-I (c1) the needle assembly 170′ and the capacitive pressure sensor assembly 150′ require double sided machining.

Referring to FIG. 11-II, unlike the fabrication process depicted in FIG. 11-I which requires double sided machining, the process depicted in this group of figures only requires top side machining. First, the handle layer is etched by deep reactive ion etching (DRIE) to form an intrusion in the silicon surrounding the membrane, FIG. 11-II (a2). Then the buried oxide layer is removed, FIG. 11-II (b2) to form a capacitive pressure sensor assembly 150″. A flat portion of the capacitive pressure sensor assembly 150″ from one side and a prefabricated insertion needle assembly 170″ on the other side are bonded, FIG. 11-II (c2). Bonding can be performed using anodic bonding or using an adhesive. Similar to the process depicted in FIG. 11-I, the process depicted in FIG. 11-II is suitable for batch fabrication.

Referring to FIG. 11-III, an insertion needle assembly 170′″ is directly attached to the backside of the silicon substrate. The process starts with DRIE to provide an access hole in the handle layer, FIG. 11-III (a3), followed by another DRIE step to provide a seat for the insertion needle assembly 170′″, FIG. 11-III (b3), to generate a capacitive pressure sensor assembly 150′″. Next, the buried oxide is removed, FIG. 12-III (c3), and the insertion needle assembly 170′″ is bonded to the capacitive pressure sensor assembly 150′″, FIG. 11-III (d3).

Referring to FIG. 11-IV, an insertion needle assembly is grown using a Vapor-Liquid-Solid (VLS) technique. Using this technique a one-dimensional object, such as a needle, can be grown. The process begins with providing an access hole using DRIE, FIG. 11-IV (a4), followed by an oxide removal step, FIG. 11-IV (b4). A thin (1-10 nm) layer of gold (Au) is patterned around the periphery of the access hole, FIG. 11-IV (c4). Then the wafer is annealed at the temperature higher than gold- silicon (Au—Si) eutectic point, providing Au—Si droplets on the surface. Next, a one dimensional crystalline needle is grown by a liquid metal alloy droplet-catalyzed chemical or physical vapor deposition process, FIG. 11-IV (d4). Au—Si droplets on the surface of the substrate act to lower the activation energy, which allows Si deposition at lower temperatures. Hence, depending on the chemicals used, at specific controlled temperature silicon can only grow below the gold covered surfaces.

### Fabrication of Coil Assembly 200 and Attaching to the Pressure Sensor

The coil assembly can be fabricated using a standard flex circuit fabrication process. Usage of a flexible material for the coil reduces the irritation for the patient and allows easy placement underneath the orbital fat. The flexible material also allows a larger area for the coil which in turn increases inductance. Higher inductance reduces the resonant frequency of the LC tank thus lowering the signal loss passing through the body. Hence coil inductance and capacitance of the IPMA 100 can be designed such that these components would not require the stimulation/response circuit 300 to be excessively close to the IPMA 100. Therefore, the components of the stimulation/response circuit 300 can be positioned a comfortable distance away from the subject in order to measure the IOP. The coil assembly 200 can be bonded to the capacitive pressure sensor assembly 150 terminals 152 and 154 using flip-chip bonding techniques.

Those skilled in the art will recognize that numerous modifications can be made to the specific implementations described above. Therefore, the following claims are not to be limited to the specific embodiments illustrated and described above. The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others.

1. A pressure monitoring system, comprising:
a stimulation/response circuit configured to transmit a first signal during a stimulation mode, and receive a second signal during a response mode in response to the first signal; and
a pressure monitoring apparatus configured to receive the first signal during the stimulation mode and transmit the second signal during the response mode, wherein the pressure monitoring apparatus comprises:
a capacitive pressure sensor assembly providing a variable capacitance in response to variable pressure being applied to the capacitive pressure sensor assembly;
an insertion needle assembly having an insertion opening, the insertion needle assembly being coupled to the capacitive pressure sensor assembly, wherein fluid pressure at the insertion opening is fluidly communicated with the capacitive pressure sensor assembly thereby affecting capacitance of the capacitive pressure sensor assembly; and
a coil assembly having an inductance, the coil assembly being coupled to the capacitive pressure sensor assembly;
wherein the coil assembly and the capacitive pressure sensor assembly form a tank circuit with a variable resonant frequency, and wherein the coil assembly receives the first signal and transmits the second signal a time difference later.

2. The pressure monitoring system of claim 1, wherein the magnitude of the second signal is dependent on the frequency of the first signal.

3. The pressure monitoring system of claim 2, wherein the magnitude of the second signal approaches a maximum when the frequency of the first signal approaches the resonant frequency of the tank circuit.

4. The pressure monitoring system of claim 3, wherein the resonant frequency of the tank circuit is based on the inductance of the coil assembly and the capacitance of the capacitive pressure sensor assembly.

5. The pressure monitoring system of claim 1, wherein the stimulation/response circuit is configured to vary frequency of the first signal to provide a frequency sweep about an expected resonant frequency of the tank circuit.

6. The pressure monitoring system of claim 1, further comprising:
a processing circuit coupled to the stimulation/response circuit;
a memory coupled to the processing circuit; and
an input/output device coupled to the processing circuit,
wherein data is communicated between the stimulation/response circuit and the processing circuit.

7. The pressure monitoring system of claim 1, wherein the pressure monitoring apparatus is configured to be implanted into a subject's eye.

8. The pressure monitoring system of claim 7, wherein the pressure monitoring apparatus is configured to transmit the second signal in response to the first signal for an elongated period of time.

9. A pressure monitoring apparatus, comprising:
a capacitive pressure sensor assembly providing a variable capacitance in response to variable pressure being applied to the capacitive pressure sensor assembly;
an insertion needle assembly having an insertion opening, the insertion needle assembly being coupled to the capacitive pressure sensor assembly, wherein fluid pressure at the insertion opening is fluidly communicated with the capacitive pressure sensor assembly thereby affecting capacitance of the capacitive pressure sensor assembly; and
a coil assembly having an inductance, the coil assembly being coupled to the capacitive pressure sensor assembly.

10. The pressure monitoring apparatus of claim 9, wherein the coil assembly and the capacitive pressure sensor assembly form a tank circuit with a variable resonant frequency, wherein the coil assembly is configured to receive a first signal, energize the tank circuit, and transmits a second signal a time difference later.

11. The pressure monitoring apparatus of claim 10, wherein the magnitude of the second signal approaches a maximum when the frequency of the first signal approaches a resonant frequency of the tank circuit.

12. The pressure monitoring apparatus of claim 11, wherein the resonant frequency of the tank circuit is based on the inductance of the coil assembly and the capacitance of the capacitive pressure sensor assembly.

13. The pressure monitoring apparatus of claim 9, wherein the capacitive pressure sensor assembly comprises:
a deformable membrane forming a first plate of a capacitor;
a second plate;
a dielectric formed between the first plate and the second plate;
wherein the insertion needle assembly is coupled to the first plate.

14. The pressure monitoring apparatus of claim 9 configured to be implanted into a subject's eye.

15. The pressure monitoring apparatus of claim 14, wherein the pressure monitoring apparatus is configured to transmit the second signal in response to the first signal for an elongated period of time.

16. The pressure monitoring apparatus of claim 9, wherein the magnitude of the second signal is dependent on the frequency of the first signal.

17. A method for monitoring intraocular pressure, comprising:
implanting a pressure monitoring apparatus in a subject's eye, the pressure monitoring apparatus having a variable capacitance in response to intraocular pressure, and an inductance forming a tank circuit;
fluidly coupling intraocular fluid with the pressure monitoring apparatus;
transmitting a first signal during a stimulation mode;
receiving the first signal by the tank circuit during the stimulation mode;
energizing the tank circuit in response to receiving the first signal; and
transmitting a second signal by the tank circuit a period of time after receiving the first signal.

18. The method of claim 17, wherein the magnitude of the second signal approaches a maximum when the frequency of the first signal approaches a resonant frequency of the tank circuit.

19. The method of claim 18, wherein the resonant frequency of the tank circuit is based on the inductance and the capacitance of the tank circuit.

20. The method of claim 17, wherein the step of implanting the pressure monitoring apparatus in a subject's eye is accomplished by an insertion apparatus, wherein the insertion apparatus is preloaded with the pressure monitoring apparatus.
